(19) United States
(12) Patent Application Publication
(10) Pub. No.: US 2020/0063108 A1
(43) Pub. Date: Feb. 27, 2020

(54) THREE-DIMENSIONAL TISSUE STRUCTURES

(71) Applicant: Dana-Farber Cancer Institute, Inc., Boston, MA (US)

(72) Inventors: Eugen DHIMOLEA, Malden, MA (US); Constantine MITSIADES, Boston, MA (US)

(21) Appl. No.: 16/489,142

(22) PCT Filed: Feb. 28, 2018
(86) PCT No.: PCT/US2018/020325

§ 371 Date: Aug. 27, 2019

Related U.S. Application Data
(60) Provisional application No. 62/464,612, filed on Feb. 28, 2017.

(51) Int. Cl. C12N 5/09; C12N 5/00; G01N 33/50; A61K 39/00

(52) U.S. Cl. CPC C12N 5/0693; C12N 5/0062; C12N 2501/40; A61K 39/0011; C12N 2513/00; G01N 33/5011

(57) ABSTRACT

The present invention provides methods of three dimensional tissue cultures and methods of using same.

This application claims priority to, and the benefit of, U.S. Provisional Application No. 62/464,612 filed on Feb. 28, 2017, the contents of which is incorporated herein by reference in its entirety.

### GOVERNMENT INTEREST

This invention was made with government support under [ ] awarded by the [ ]. The government has certain rights in the invention.

### FIELD OF THE INVENTION

The present invention relates generally to three dimensional tissue culture systems as a model for post-treatment residual disease and methods of using same.

### BACKGROUND OF THE INVENTION

Tissue engineering is the use of a combination of cells, engineering, materials and methods, as well as suitable biochemical (e.g., growth factors) and physico-chemical factors (e.g., chemically-modified extracellular matrices) to improve, replace or mimic biological structures and/or functions. Tissue engineering is widely accepted as an interdisciplinary field that applies the principles of engineering and life sciences toward the development of biological substitutes that restore, maintain, or improve tissue function or a whole organ. Engineered tissue systems not only have significant potential in the area of regenerative medicine to restore and/or repair damage or diseased tissues, but have also been proposed for use in drug discovery and development as providing access to more accurate and physiologically relevant model systems for predicting and/or testing the pharmacokinetic and pharmacodynamic responses associated with pharmacologic agents.

Among the major challenges facing tissue engineering is the need for more complex and physiologically relevant engineered tissues that better mimic the structure, physiology, and function, of native tissues. This is particularly important and challenging when attempting to use engineered tissues to screen, test, and/or evaluate therapeutic agents.

Tissue engineering provided three-dimensional biological tissues that accurately mimic native physiology, architecture, and other properties of native tissues can be used to effectively, reliably, and accurately evaluate the interaction and effects of pharmacologic agents on a subject.

Engineered tissue model systems for cancer, in particular drug refractory cancer would improve the arduous drug development and discovery process. Such a need exists in the art. The present disclosure provides various solutions to these art-recognized problems by providing methods, compositions, and devices for using three-dimensional biological tissues to generate cells that accurately mimic native physiology, architecture, and other properties of cancer cells that survive in tumor tissues after treatment with a therapeutic agent (post-treatment residual cancer cells) by resisting the cytotoxic effect of the given agent, for use in, among other applications, drug testing, personalized disease treatment, regenerative medicine or combinations thereof.

### SUMMARY OF THE INVENTION

In various aspects, the invention provides methods of preparing a drug refractory cell or organoid by culturing a population of cells in a three-dimensional cell culture system in the presence of one or more agents capable of inducing death of the cells for a period of time until the longitudinal rate of decreasing cell viability in the culture approximately plateaus which is otherwise defined by the inflection time point after which the slope of the longitudinal segment of the cell viability curve is the closest to zero

The invention further provides methods of preparing a cell or organoid in a diapause like state by culturing a population of cells in a three-dimensional cell culture system in the presence of one or more agents capable for a period of time until the longitudinal rate of cell growth or of decreasing cell viability in the culture approximately plateaus which is otherwise defined by the inflection time point after which the slope of the longitudinal segment of the cell viability curve is the closest to zero

The agent is for example, a cytotoxic agent or a targeted therapeutic agent. A cytotoxic agent includes but is not limited to irradiation. Alternatively, the agent is a cell stress inducing agent. The targeted agent is an antibody, a peptide or a nucleic acid.

Optionally, the methods further includes culturing the cells in the presence of a one or more developmental morphogens. A developmental morphogen is for example, a Wnt pathway stimulator such as R-spondin or Noggin.

In other aspects, the population of cells are transfected with an expression vector encoding a reporter protein prior to culturing in the three-dimensional cell culture system.

The cell or organoid is any type of cell. The cell or organoid is a tumor. The populations of cells is a primary tumor cell or a tumor cell line. Alternatively, the populations of cells is a tumor of human or animal origin growing in an animal.

Also included in the invention is the drug refractory or diapause-like cell or organoid produced by methods of the invention

In another aspect, the invention provides a method of screening a candidate drug, or a candidate drug combination for anti-cancer activity by contacting the cell or organoid produced according to the invention with the candidate drug or drug combination and determining whether the candidate induces cell or organoid death or inhibits cell or organoid growth.

In yet another aspect, the invention provides method of longitudinally simulating the relapse of residual cancer in a subject by longitudinal measurement of growth of the cell or organoid produced according to the invention

In a further aspect the invention provides a method of screening a candidate drug, or a candidate drug combination capable of treating residual cancer in a subject by contacting the cell or organoid produced according to the invention with a candidate drug or drug combination and determining whether the candidate induces cell or organoid death or inhibits cell or organoid growth. The cell or organoid is autologous to the subject.

In another aspect the invention provides a method of screening a candidate drug, or a candidate drug combination capable of capable or reversing the drug refractory state or the diapause like state, by contacting the cell or organoid produced according to the invention with a candidate drug or drug combination and determining if the candidate drug is capable or reversing the drug refractory state or the diapause like state.

Growth or inhibition of growth of the cell or organoid is determined for example, by a cell viability assay, a cell reporter assay, or a microscopic assay.

The candidate drug combination is administered concomitantly or sequentially.

The various aspects the invention provides a cancer vaccine including the cell or organoid produced according to the invention or portion thereof. The cell or organoid is dead, irradiated and/or modified to express an immune-stimulatory factor.

The invention further provides a method of identifying a biomarker associated with a drug refractory tumor or a diapause like state, by comparing a gene or protein expression profile obtained from the cell or organoid produced according to the invention, with a reference gene or protein expression profile obtained from cell population used to produce the organoid.

In a further aspect the invention provides a cancer vaccine composition containing the cell or organoid produced according to the invention. The cell or organoid is dead, irradiated and or modified to express an immune-stimulatory factor.

In yet another aspect the invention provides a regenerative medicine method using the cell or organoid produced according to the invention. The cell or organoid contains desirable gene expression, epigenetic, and/or stem cell-like properties.

Unless otherwise defined, all technical and scientific terms used herein have the same meaning as commonly understood by one of ordinary skill in the art to which this invention pertains. Although methods and materials similar or equivalent to those described herein can be used in the practice of the present invention, suitable methods and materials are described below. All publications, patent applications, patents, and other references mentioned herein are expressly incorporated by reference in their entirety. In cases of conflict, the present specification, including definitions, will control. In addition, the materials, methods, and examples described herein are illustrative only and are not intended to be limiting.

Other features and advantages of the invention will be apparent from and encompassed by the following detailed description and claims.

### BRIEF DESCRIPTION OF THE DRAWINGS

FIG. 1: Right: Longitudinal exposure of MDA-MB-231 3-D organoids and 2-D cultures to cytotoxic drugs (100 nM). Exposure to docetaxel kills virtually all the cells in the 2D culture but only a fraction of the cells in the 3D culture spheroids; plateauing of the viability curve indicates emergence of drug-refractory organoids. Left: H&E staining of day-15 drug-refractory MDA-MB-231 organoids and respective control.

FIG. 2. Left: Exposure of prostate cancer patient-derived 3-D organoids, cultured in 3D conditions in the presence of Wnt-stimulator R-spondin, to cytotoxic drugs (100 nM; vinblastine highlighted with red) and validation of vinblastine response in the respective patient-derived xenograft (PDX) model. Notice cell viability curve plateauing in both in vitro and in vivo models, indicating emergence of drug-refractory organoids and residual tumors, respectively. Right: Histological similarities of in-vitro vs. in-vivo drug-refractory malignant foci.

FIG. 3: Longitudinal (time-lapse) response of patient-derived breast cancer 3D Organoids, cultured in 3D conditions in the presence of Wnt-stimulator R-spondin, to multiple approved and investigational anti-cancer agents including common chemotherapeutics (100 nM) and various classes of kinase inhibitors (100 nM). Exposure to most active anti-cancer compounds leads to fractional cell killing of variable magnitudes and eventual emergence of drug-refractory 3D organoids as indicated by the plateau phases of the respective curves.

FIG. 4: A) Right: Similar longitudinal drug-response dynamics of breast cancer PDX and the respective 3D organoid culture (cultured in 3D conditions in the presence of Wnt-stimulator R-spondin) to the kinase inhibitor Afatinib, resulting in adapted, drug-refractory, residual cancer cells in both models. Notice cell viability curve plateauing in both in vitro and in vivo models, indicating emergence of drug-refractory organoids and residual tumors, respectively. B) Overlap of informative genes with statistically significant change in both the PDX (left) and 3D Organoids (right), after Afatinib treatment (respective residual disease models). Out of 335 genes upregulated in PDX, 255 were also upregulated in 3D Organoids; and out of 419 genes downregulated in PDX, 390 were also downregulated in 3D Organoids (Fisher exact test p<0.0001). Highlight of expression changes in various genes/gene families indicating similar drug-induced molecular adaptation processes in PDX vs. 3D Organoid models, and including the emergence of potentially druggable targets.

FIG. 5: A) Examples of molecular network up regulated in drug-refractory patient-derived 3D Organoids after exposure to Docetaxel (100 nM). B) Example of biomarker expression changes in drug-refractory patient-derived 3D Organoids after exposure to Docetaxel (100 nM)

FIG. 6: 2D annotation enrichment analysis' of transcriptional changes of Diapause/E4.5 epiblasts and Docetaxel-refractory/Vehicle MDAMB231 3-D organoids, across two public molecular databases: ConsensusPathDB (CPDB) and Gene Ontology Consortium (GO); Spearman correlation scores indicated on the graph.

FIG. 7: 2D annotation enrichment analysis' of transcriptional changes of Diapause/E4.5 epiblasts and Docetaxel-refractory/Vehicle breast cancer patient-derived 3-D organoids, cultured in 3D conditions in the presence of Wnt-stimulator R-spondin, across two public molecular databases: ConsensusPathDB (CPDB) and Gene Ontology Consortium (GO); Spearman correlation scores indicated on the graph.

FIG. 8: Examples of pathways commonly upregulated/downregulated in drug-refractory organoids and embryonic diapause (derived from pairwise annotation enrichment analysis).

FIG. 9: Scatterplot of gene expression changes in publicly available dataset of BrCa clinical residual disease after taxane treatment and in docetaxel-refractory MDAMB-231 3-D organoids.

FIG. 10: Afatinib-refractory and vinblastine-refractory prostate cancer 3-D organoids longitudinally exposed to afatinib and vinblastine (100 nM) 5 days after the first exposure drug-washout. Notice cross-resistance in vinblastine-refractory 3D organoids and lack thereof in afatinib-refractory 3D organoids.

FIG. 11: Breast cancer patient-derived 3-D organoids treated with DMSO-control (left) or 100 nM Docetaxel (center) for 10 days, and 8 weeks after docetaxel washout (right). Notice the ability of drug-refractory organoid to regenerate fully-grown organoids.

FIG. 12: A) Protein expression ad phosphorylation changes in breast cancer patient-derived drug-refractory organoids are partially reversed after drug withdrawal (comparison with vehicle-control). B) Gene expression changes in breast cancer patient-derived drug-refractory organoids are partially reversed after drug withdrawal (normalized RPKM counts). C) Distinct gene expression changes induced by docetaxel and afatinib on the tubulin and glutathione-S-transferases gene families in drug-refractory breast cancer 3-D organoids (comparison with the DMSO control).

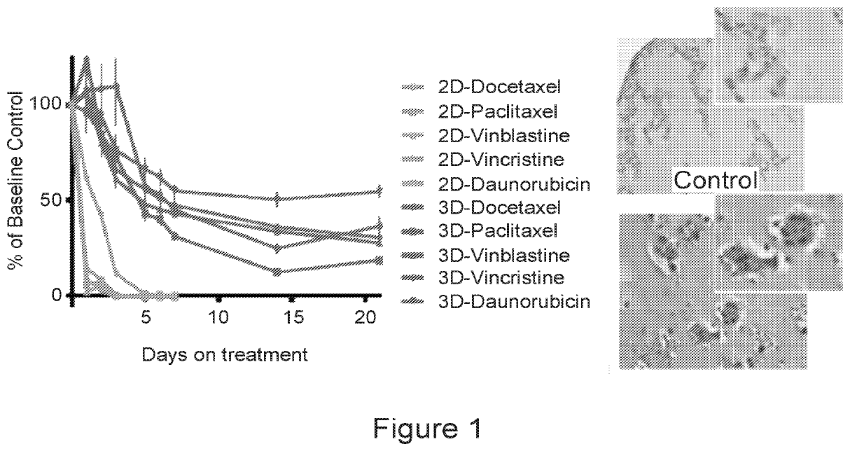

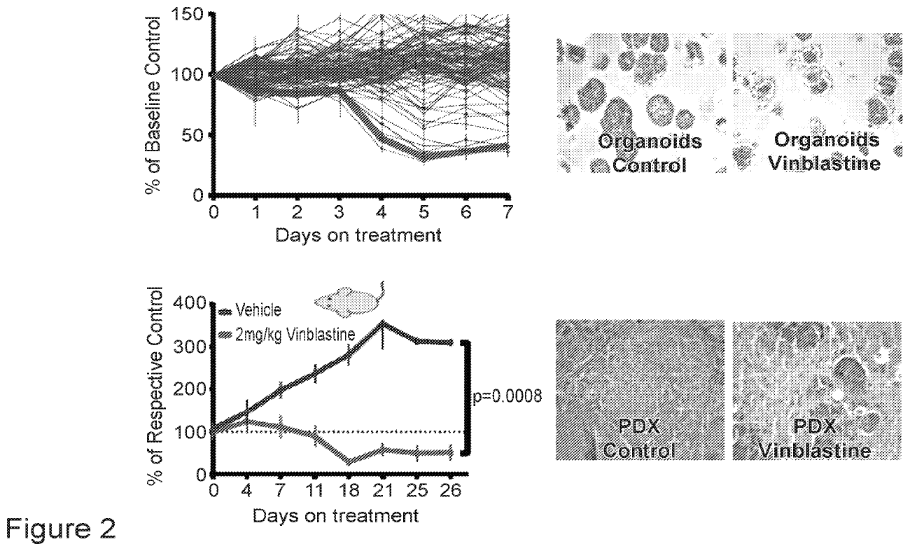

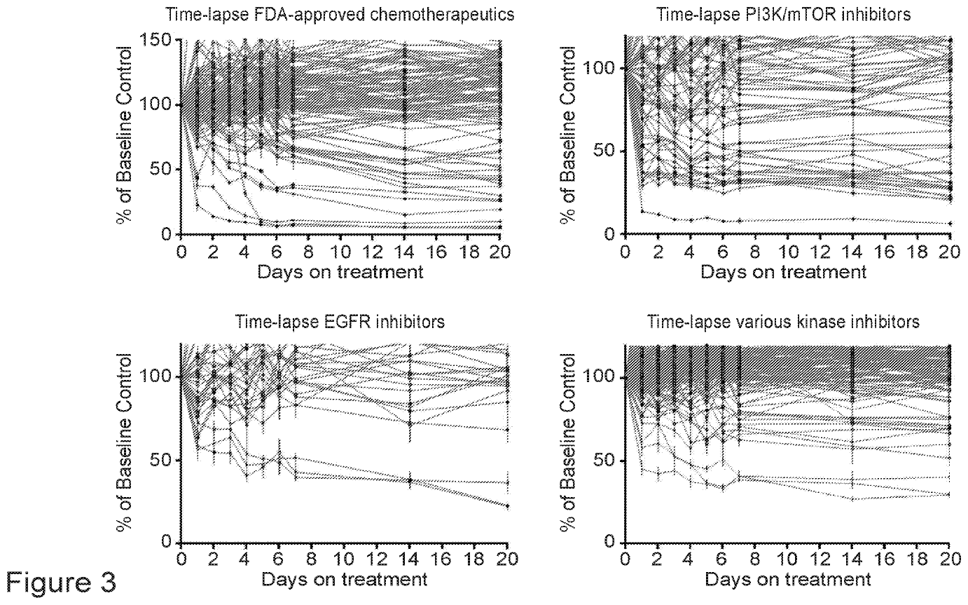

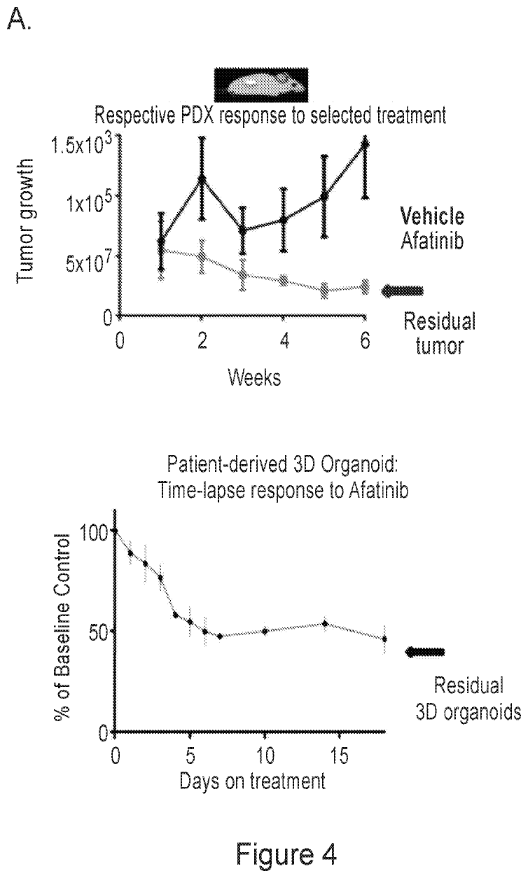

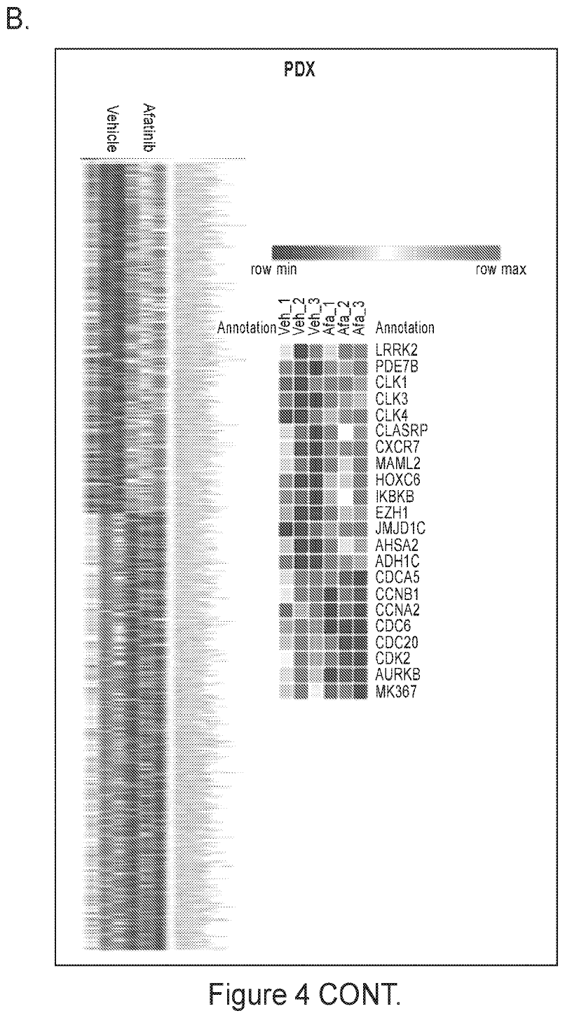

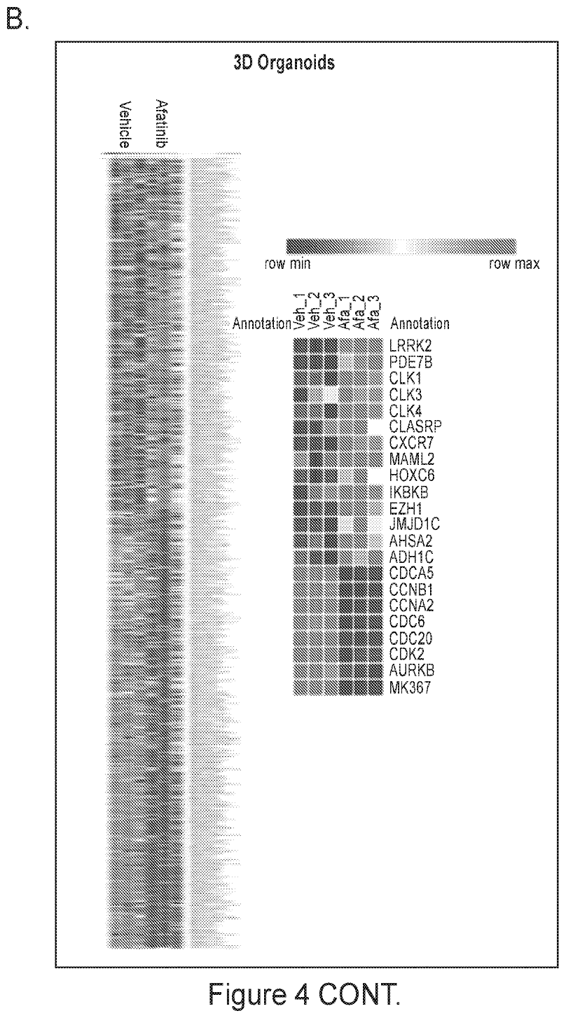

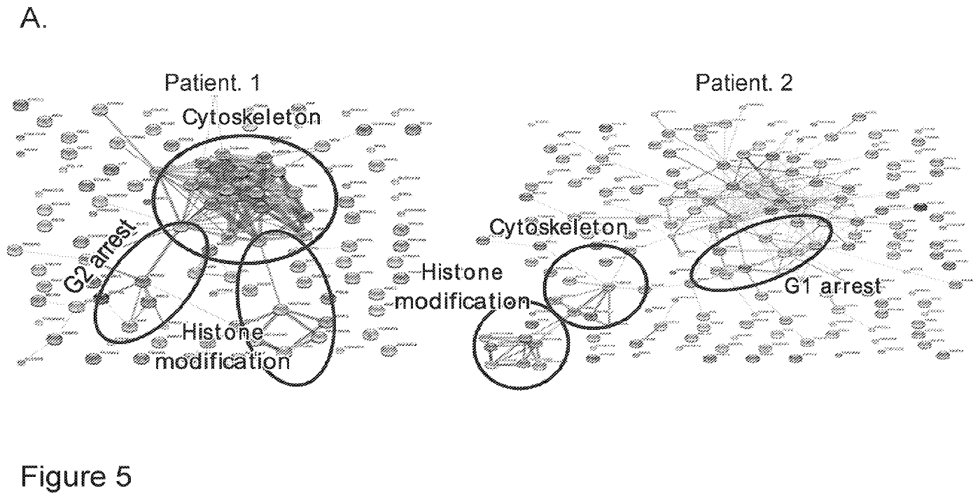

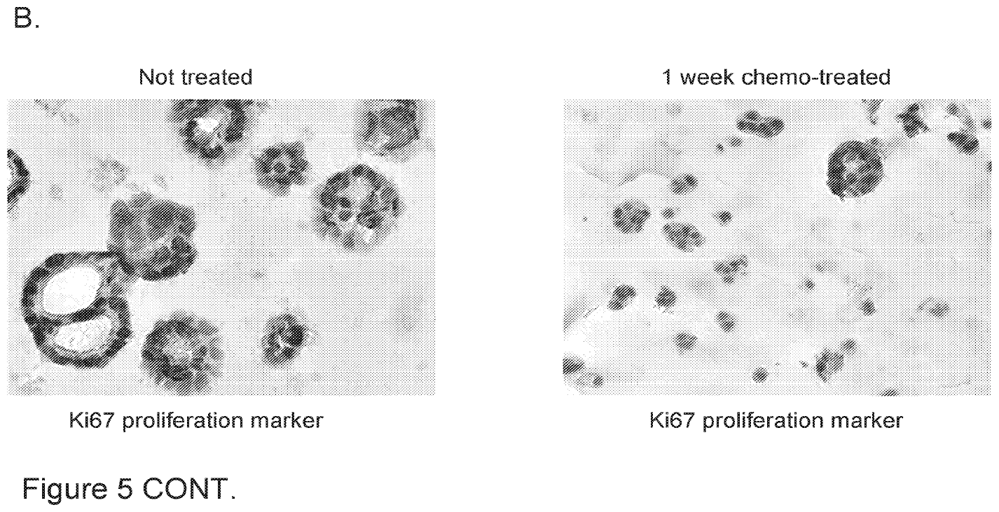

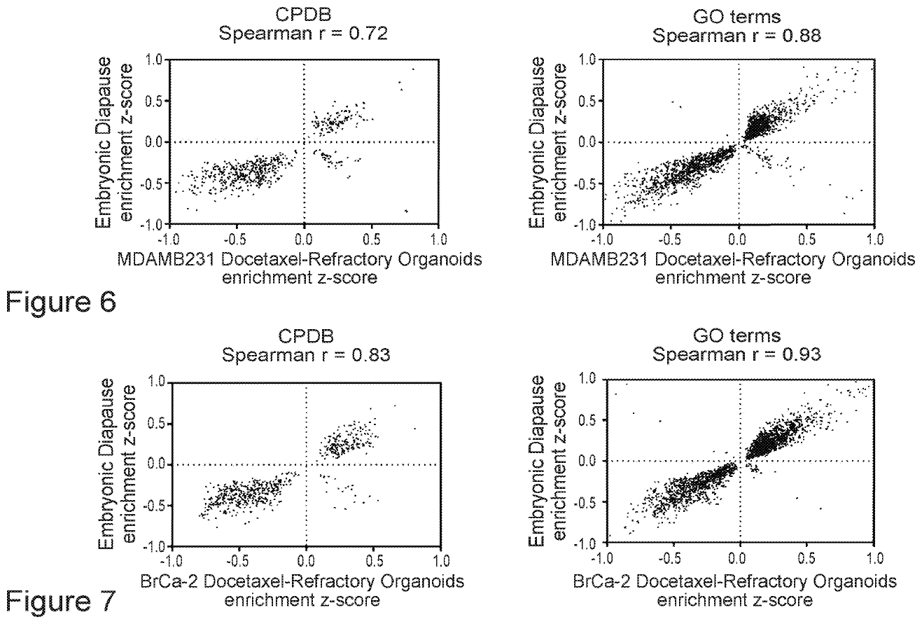

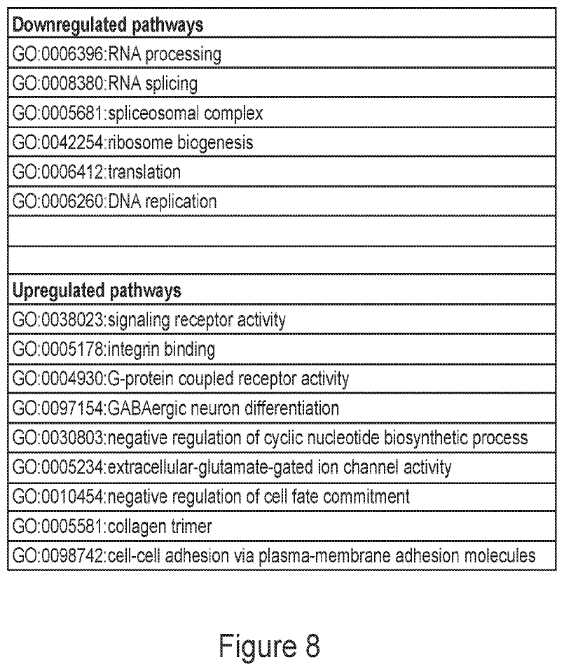

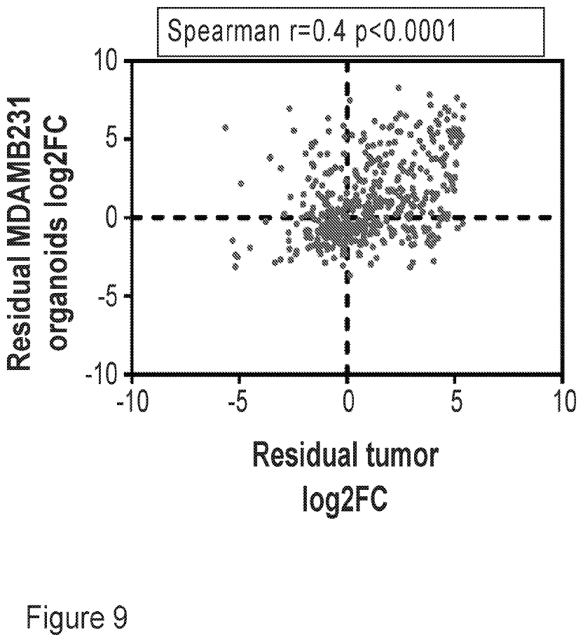

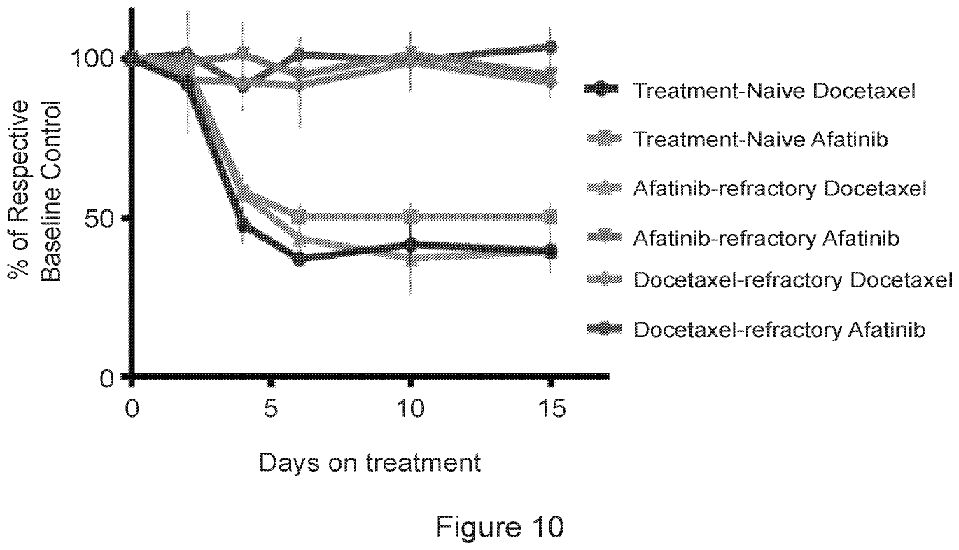

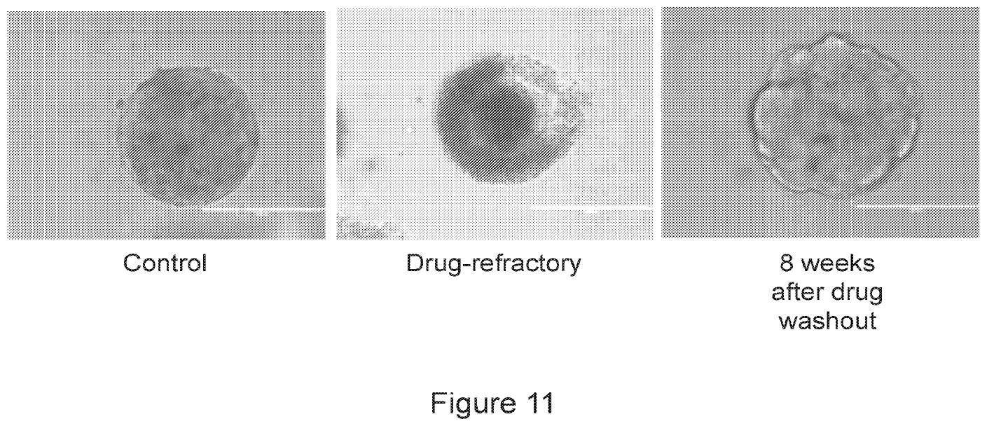

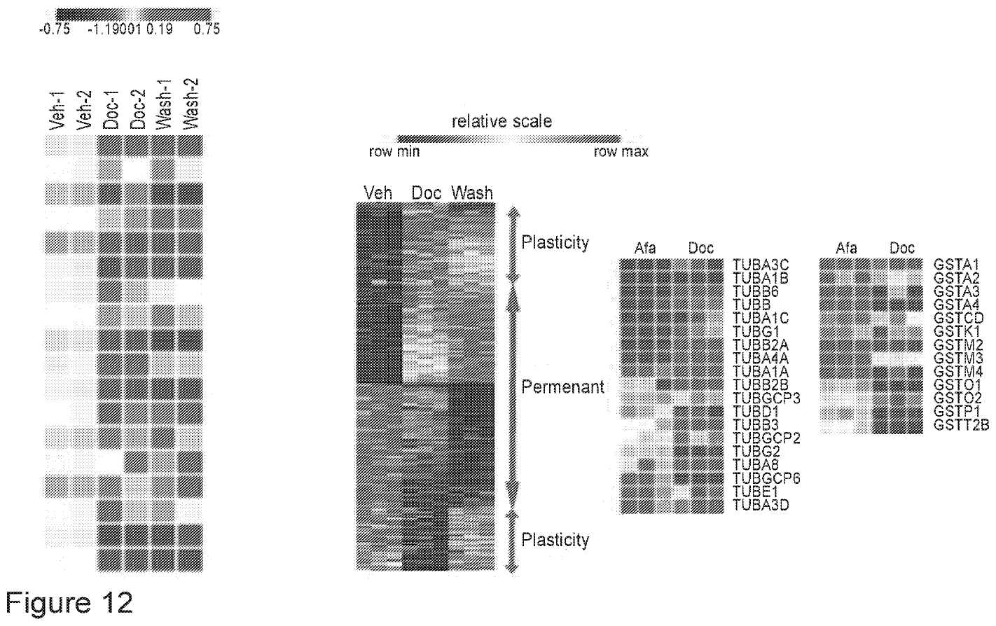

### DETAILED DESCRIPTION OF THE INVENTION

The invention is based in part upon the discovery of cell cultures methods that generate populations of post-treatment cancer cells that are refractory to drug-induced cytoxicity and generate populations of cells that are in a embryonic diapause like state. Specifically, it has been discovered that cancer cell lines and cancer patient derived cells grown as organoids in 3D cultures (but not 2D cultures) in the presence of serum and/or agents that stimulate developmental pathways and cytotoxic or targeted drugs generate surviving cancer organoid subpopulations that are refractory to drug-induced cytoxicity.

Given the growing recognition and acceptance of the important role that tumor microenvironment plays in cancer formation and progression, a three dimensional (3D) culture system has been developed that recreates the molecular and biomarker profile, morphology and drug refractory phenotypes of post-treatment residual disease.

The cancer cells produced in this way show morphological and biochemical properties similar to those shown by post treatment residual disease in vivo, and are different from cancer cells grown in two-dimensional cultures. Cancer cells cultured in accordance to methods of the invention mimic the drug refractory phenotype of post treatment residual disease, allowing short- and long-term study of the adapted drug refractory state. This system provides a microenvironment more relevant to the living animal than traditional two-dimensional culture systems.

Additionally, the cells produced in this way are genetically similar to cells that are in an embryonic diapause state. Specifically, the 3D culture systems according to the invention produces cells or organoids that share pathways commonly upregulated or downregulated during embryonic diapause, a reversible, suspended development state. This suggests that the cell in the drug refractory state is a suspended developmental state similar to cells such as epiblasts in diapause. As diapause is triggered by environmental stressors and is reversible, the cells or organoids produced by the invention can be used to test deprogramming or reversal of the refractory state.

The 3D culture system according to the invention has several advantages, including, but not limited to: (i) it does not require cumbersome protocols of biomarker-based isolation, such as by flow cytometry; (ii) it does not require genetic manipulation of immortalized cell populations in order to increase the stem-like cell content; (iii) it is entirely conducted in ECM-based 3D cultures which increases its biological relevance; (iv) it can accommodate the inclusion of stromal and/or components, which increases it biological relevance; (v) it generates cancer 3D organoids that are fully refractory to existing known anticancer drugs, for prolonged time periods; and (vi) it is compatible with 3D culture methods known to enable expansion of patient-derived cells.

The 3D culture system has several areas of application, including, but not limited to: (i) it can be used to culture cancer cells alone and study their responses to bioactive molecules and therapeutic drugs; (ii) it can be used to study interactions between cancer cells and other cell types; (iii) it can be used to mimic microenvironments in which cancer cells, specifically drug refractory cancer cells develop; (iv) it can be used to identify prognostics biomarkers; (v) it can be used to identify new targets for existing drugs; (vi) it can be used to identify drugs capable of inducing cell death or inhibiting the growth of drug refractory cells; (vii) it can be used to mimic microenvironments in a diapause state develop; (viii) it can be used to identify biomarkers for the diapause-like state; (ix) it can be used to identify new targets for existing drugs; (xi) it can be used to identify drugs capable reversing the drug refractory state or diapause like state; (xii) it can be used to generate cancer vaccine against post-treatment residual cancer; and (xii) it can be used to generate components useful in regenerative medicine.

The 3D culturing system and methods of the invention represents a significant advancement for the study of cancer cells in a context that more closely mimics residual disease in cancer patients. This cell culture system provides a novel screening method for ex vivo characterization of tumors in individual cancer patients and determining the response of such tumors to single agent or combination drug treatments. This high-throughput 3D culture system can be used to rapidly assess the response of cancerous tissue biopsies to different combinations of therapeutic regimens thereby allowing for the rapid and rational selection of a specific combination of drugs for personalized and highly effective cancer therapy.

This 3D cell culture system further provides a novel screening method for evaluating compounds with potential anti-cancer activity or the potential to reverse the drug refractory state. This high-throughput 3D culture system can be used to rapidly assess the response of cancer cell lines or cancerous tissue biopsies to new compounds or combination of compounds thereby allowing for the rapid identification of new therapeutic agents useful for treating cancer, in particular, treating post-treatment residual disease.

The 3D culture system and methods of the present invention is amenable to testing and screening any potential therapeutic strategy, ranging from small molecular weight inhibitors, to monoclonal antibodies, to siRNA-based, microRNA-based and vector-based therapies, immunotoxin or nanoparticle-based drug delivery, and to various combinations.

This 3D cell culture system further provides a method for identifying biomarker or biomarker profiles useful as prognostic and diagnostic tools. In particular, the biomarkers identified using the cell culture system of the invention are useful in determining the responsiveness, e.g., sensitivity or resistance, of a cancer cell to treatment. These biomarkers are also useful for monitoring subjects undergoing treatments and therapies for cancer and for selecting therapies and treatments that would be efficacious in subjects having cancer.

This 3D cell culture system further provides a method for identifying new targets for existing drug, in particular the identification of drugs that will be effective in treating drug refractory cancer cells and/or reversing the drug refractory state.

In other aspects, the 3D cell culture system allows for the generation of cancer vaccine against post-treatment residual disease. For example, the drug refractory cells or organoids produced by the methods of the invention can be used (dead, irradiated or parts thereof) as components of a vaccine composition to target post-treatment residual cancer.

In a further aspect, the 3D cell culture system allows for the generation of components, such as epigenetically modified epithelial cells, useful in regenerative medicine.

This 3D culture system is amenable and well-suited to grow cells that would include but not be limited to human and other mammalian cell lines, primary and secondary cultures for animal models of cancer or diapause, cell preparations obtained by fine needle aspirates of, core needle biopsies, brushed exfoliated cells, incisional or exiscional biopsies and surgically resected tissue from solid tumors, as well as normal epithelial cells of human and animal origin.

The 3D cultures of the invention may be produced by any 3D culturing methods know in the art. For example, the 3D culturing methods may utilize scaffold techniques or scaffold-free techniques.

Scaffold techniques include the use of solid scaffolds, hydrogels and other materials. Hydrogels are composed of interconnected pores with high water retention, which enables efficient transport of e.g. nutrients and gases. Several different types of hydrogels from natural and synthetic materials are available for 3D cell culture, including e.g. animal ECM extract hydrogels, protein hydrogels, peptide hydrogels, polymer hydrogels, and wood-based nanocellulose hydrogel.

Scaffold free techniques employ another approach independent from the use scaffold. Scaffold-free methods include for example the use of low adhesion plates, hanging drop plates, micropatterned surfaces, and rotating bioreactors, magnetic levitation, and magnetic 3D bioprinting.

The present invention relates to methods of producing drug refractory organoids. In some aspects, the drug refractory organoids are produced by providing a suspension of cells that is prepared from at least one biological tissue and/or cell-containing bodily fluid in a medium. Preferably, the biological tissue and or bodily fluid is obtained from a subject having cancer.

The present invention also relates to methods of producing cells and organoids in a diapause like state. In some aspects, the cells or organoids in a diapause like state are produced by providing a suspension of cells that is prepared from at least one biological tissue and/or cell-containing bodily fluid in a medium.

The subject is a human. Alternatively, the subject is an animal bearing a cancer of human or animal origin. Preferably the animal bearing the cancer of human or animal origin is an immunocompromised mouse.

The subject has not received treatment for the cancer. Alternatively, the subject has received treatment for the cancer.

The concentration of cells in the suspension of single cells is adjusted and optionally an inert matrix is then added to the suspension of single cells, which is then incubated, preferably in the presence of CO₂.

In the process according to the invention the cells of the biological tissue and/or cell containing bodily fluid are first dissociated or separated from each other. Dissociation of the tissue is accomplished by any conventional means known to those skilled in the art. Preferably the tissue is treated mechanically or chemically, such as by treatment with enzymes. More preferably the tissue is treated both mechanically and enzymatically. Use of the term “mechanically” means that the tissue is treated to disrupt the connections between associated cells, for example, using a scalpel or scissors or by using a machine, such as a homogenizer. Use of the term “enzymatically” means that the tissue is treated using one or more enzymes to disrupt the connections between associated cells, for example, by using one or more enzymes such as collagenase, dispases, DNAse and/or hyaluronidase. Preferably a cocktail of enzymes is used under different reaction conditions, such as by incubation at 37° C. in a water bath or at room temperature with shaking.

The dissociated tissue is then suspended in a medium to produce a suspension of cells and from which the organoids can be formed directly.

Preferably the cells (patient-derived or cell line) are treated to remove dead and/or dying cells and/or cell debris. The removal of such dead and/or dying cells is accomplished by any conventional means known to those skilled in the art for example, using beads and/or antibody methods. It is known, for example, that phosphatidylserine is redistributed from the inner to the outer plasma membrane leaflet in apoptotic or dead cells. Annexin V and any of its conjugates which have a high affinity for phosphatidylserine can therefore be bound to these apoptotic or dead cells. The use of Annexin V-Biotin binding followed by binding of the biotin to streptavidin magnetic beads enables separation of apoptotic cells from living cells. Preferably the patient-derived cancer cells are treated to remove non-epithelial cells such as blood cells and/or fibroblasts. The removal of those cells can be accomplished with methods similar to the ones described above and included bead and/or antibody methods of positive selection (for epithelial cells) and negative selection/depletion for non-epithelial cells. Other suitable methods will be apparent to the skilled artisan.

The suspension of cells is prepared in a culture medium. The medium is designed such that it is able to provide those components that are necessary for the survival of the cells. Preferably the suspension of single cells is prepared in a medium comprising one or more of the following components: serum, buffer, chemokines, growth factors, hydrogen carbonate, glucose, physiological salts, amino acids and hormones.

A preferred medium is DMEM/F12. DMEM/F12 medium has been used for the culture of human normal and neoplastic epithelial cells. DMEM/F12, when properly supplemented with cytokine stimulator of the WNT pathway, including but not limited to R-spondin and Noggin, has demonstrated wide applicability for supporting growth of many types of primary normal or neoplastic epithelial cells and cell lines.

Preferably, the medium further comprises L-glutamine, in particular a stabilized L-glutamine. L-glutamine is an essential nutrient in cell cultures for energy production as well as protein and nucleic acid synthesis. However, L-glutamine in cell culture media may spontaneously degrade forming ammonia as a by-product. Ammonia is toxic to cells and can affect protein glycosylation and cell viability, lowering protein production and changing glycosylation patterns. It is thus preferred that the L-glutamine is a stabilized glutamine, most preferably it is the dipeptide L-alanyl-L-glutamine, which prevents degradation and ammonia build-up even during long-term cultures. The dipeptide is commercially available as GlutamaxI™ (Invitrogen, Carlsbad, Calif.).

The medium may further comprise additional components such as antibiotics, for example, penicillin, streptomycin, neomycin, ampicillin, metronidazole, ciprofloxacin, gentamicin, amphotericin B, kanamycin, nystatin; amino acids such as methionine or thymidine; FCS and the like.

In addition to, or instead of, DMEM/F12 other liquid media can be used, for example RPMI1640, Ham's F-10, McCOY's 5A, F-15, RPMI high or low glucose, Medium 199 with Earle's Salts or the different variants of MEM Medium.

The concentration of cells in the suspension of single cells is adjusted to an appropriate cell concentration. An appropriate cell concentration means an amount of cells per milliliter of culture medium which supports the formation of organoids in the incubation step. Appropriate cell amounts are preferably 10³ to 10⁷ cells/ml medium, more preferably 10³ to 5×10⁶ cells/ml medium and most preferred 10⁵ to 10⁶ cells/ml medium. Methods of determining cell concentration are known in the art, for example, the cells may be counted with a Neubauer counter chamber (hemocytometer).

The cells are used to generate 3-dimensional (3D) cultures. The 3D cultures are preferably generated by suspending the cells scaffold matrix or parts thereof. Alternatively the 3D culture is generated by aggregating the cells using techniques known to persons skilled in the art including low-attachment plates, hanging drops and magnetic bead-based aggregation methods.

In the preferred method for generating the 3D cultures an appropriate amount of a scaffold material is added to the suspension of cells. Use of the term scaffold as used herein refers to a matrix. Preferentially the matrix is a hydrogel. The preferred hydrogel scaffold matrix is comprised of one or more molecules of extracellular matrix of human or animal origin in their native form or modified. Alternatively the hydrogel scaffold can be comprised of artificially synthesized molecules that contain peptide domains corresponding to extracellular matrix molecules. Alternatively the scaffold can be comprised of inert material that has only limited or no ability to react chemically and/or biologically, i.e., having little or no effect on the biological behaviour/activity of the cells of the suspension of single cells.

Thus, the scaffold matrix supports or aids the formation of spheroids during the incubation step. Preferably the scaffold matrix is added to the culture medium in an amount of 50 to 90% vol. % based on the total volume of the medium. The extracellular matrix based scaffold preferably contains molecules of laminin, collagen, fibronectin and other molecules contained in natural extracellular matrices of human or animal origin, or mixtures thereof. Preferred materials for extracellular matrix based scaffolds are extracts of Engelbreth-Holm-Swarm murine tumors (matrigel or variants thereof), laminin-based extracellular matrix, collagen type I and collagen type IV. Alternatively preferred materials for the extracellular matrix based scaffold are self-assembling synthetic peptides containing aminoacid domains of extracellular matrices. A preferred aminoacid domain is the RGD domain. The inert scaffold matrix is preferably a non-ionic poly(ethylene oxide) polymer, water soluble resin or water soluble polymer such as a cellulose ether. Preferably the inert matrix is selected from the group comprising carboxymethyl cellulose, hydroxypropyl cellulose, hydroxypropylmethyl cellulose, hypomellose, methyl cellulose, methylethyl cellulose. However, also suitable is cellulose, agarose, seaplaque agarose, starch, tragacanth, guar gum, xanthan gum, polyethylene glycol, and the like.

The cell suspension is then incubated, preferably in the presence of CO₂. Incubation can also be carried out in the presence of water vapor. Possible preparation techniques are e.g., matrigel-based gels, collagen-based gels, the the liquid-overlay technique, the spinner flask technique, the high aspect rotating vessel (HARV) technique, the low attachment plates or the hanging drop method. These methods are known to the skilled artisan. The HARV technique is inter alia disclosed in U.S. Pat. Nos. 5,153,131, 5,153,132, 5,153,133, 5,155,034, and 5,155,035. The spinner flask technique is disclosed in e.g., W. Mueller-Klieser, “Multicellular Spheroids”, J. Cancer Res. Clin. Oncol., 12: 101-122, 1986. The liquid-overlay technique is disclosed e.g., in J. M. Yuhas et. al., “A simplified method for production and growth of multicellular tumor spheroids”, Cancer. Res. 37: 3639-3643, 1977. The hanging drop method is disclosed in e.g., Bulletin of Experimental Biology and Medicine, Vol. 91, 3, 1981, Springer, New York. Most preferred in the present invention are the matrigel-based and/or collagen-based gels. The cells are cultured in a medium that may contain serum, Epidermal Growth Factor, Fibroblast Growth Factor, steroid hormones, anti-oxidants such as N-Acetyl cysteine and/or glutathione, vitamins such as nicotinamide, supplements such as B27, inhibitors of the ROCK pathway, developmental morphogens. i.e., agents that stimulate developmental pathways. In a preferred application the cells are cultured the the presence of developmental morphogens that stimulate the Wnt pathway. Exemplary stimulators of the Wnt pathway include for example, the ligands R-spondin or Noggin. The culture medium further includes a cytotoxic agent or a targeted drug.

Systems and devices for three-dimensional cell culture are know in the art. For example the systems and devices described in WO2016/004015, the contents of which is incorporated by reference in its entirety.

It has surprisingly been found that organoids produced from primary isolated tissue or cell lines in the presence of serum or of a developmental morphogen and a cytotoxic agent or a targeted drug can generate can generate surviving cancer cells or cancer organoids that are virtually completely refractory to cytotoxicity induced by the said cytotoxic agent or targeted drug even after continuous long-term exposure to the drug for up to 3 weeks. Morphological analysis of the 3D cultures demonstrates that the drug-resistant cells or organoids also undergo histological changes similar to those induced in human tumors after drug exposure in vivo. It has also been observed that the drug-resistant adaptive state of cancer cells or organoids in these conditions is accompanied with profound molecular changes, including cell cycle arrest, activation of DNA damage response, upregulation of drug-metabolizing enzymes, downregulation of activators of cell cycle etc. These molecular features are similar to those observed in vivo in drug-resistant cancer stem cells, might enable the resistant phenotype of this cell population.

Importantly, the cells and organoids demonstrate molecular changes (i.e., gene expression, gene ontology (GO), molecular or biological pathways) similar to cells in an embryonic diapause state.

In contrast, exposure of the respective 2D cultures to the same cytotoxic agent or targeted drug causes the death of virtually all the cell. This result was surprising and unexpected. Thus the systems and methods or the invention are superior to traditional 2D cultures as the 2D cultures cannot be used in short-term assays (approximately 3 days to 3 weeks) to generate similar adapted drug-refractory cancer cell populations.

In some aspects the cells (patient-derived or cell line) are labeled prior to initiating the 3D organoid cultures. For example, the cells are labeled with methods such as introduction of expression vector for genes such as luficerase or fluorescent proteins. Labeling the cells allows for practical longitudinal measurements of the cell viability and for the longitudinal determination of the time point in which the cells acquire the adapted drug-refractory state or diapause like state.

Viability is determined by methods known in the art. A preferred method to determine cell viability is the measurement of bioluminescence in cells stably transfected with the luciferase expression vector upon addition of the luciferase substrate. An alternative method to determine cell viability is the measurement of fluorescence in cells stably transfected with the expression vectors of fluorescent protein (e.g. GFP or RFP). Alternatively, the cell viability can be measured by ATP-based assays; for example the Cell-Titer Glow assay. Alternatively, the cell viability can be measured by colorimetric assays, for example the MTT assay. Alternatively, cell viability can be measured by conventional or automated microscopy methods as determined by the size and shape of the cancer organoids. Alternatively, cell viability can be measured by assays measuring mitochondrial function such as the BH3 profiling assay. Further, cell viability can be measured by other methods, such as for example dye exclusion assays (e.g., trypan blue, eosin, or propidium iodide. In the trypan blue test, a cell suspension is simply mixed with dye and then visually examined to determine whether cells take up or exclude dye. A viable cell will have a clear cytoplasm whereas a nonviable cell will have a blue cytoplasm. Dye exclusion is a simple and rapid technique measuring cell viability but it is subject to the problem that viability is being determined indirectly from cell membrane integrity. A more sophisticated method of measuring cell viability is to determine the cell's light scatter characteristics, 7AAD or propidium iodide uptake. It will be apparent to one skilled in the art that use of a flow cytometer coupled with cell sorting may also accomplish removal of dead and/or apoptotic cells.

Some aspects of this invention provide methods for identifying an effective anti-cancer agent using the drug refractory cells or organoids as provided herein. In some embodiments, the method comprises contacting the drug refractory cells or organoids as provided herein with candidate agent, for example, by adding the agent to the a culture containing the drug-refractory cells or organoids. In some embodiments, the method comprises identifying the candidate agent as an effective anti-cancer agent if the candidate agents cause cell death.

In some embodiments, the method comprises assessing a biomarker associated with the drug-refractory organoid. In some embodiments, the method comprises comparing the assessed biomarker with a reference value.

In other aspects, this invention provide methods for identifying an agent capable of reversing the diapause like state or the drug refractory states using cells or organoids as provided herein. In some embodiments, the method comprises contacting the cells or organoids as provided herein with candidate agent, for example, by adding the agent to the a culture containing the cells or organoids. In some embodiments, the method comprises identifying the candidate agent as a diapause reversing or drug refractory reversing if the candidate agents cause cell death.

In some embodiments, the method comprises assessing a biomarker associated with the diapause like state organoid. In some embodiments, the method comprises comparing the assessed biomarker with a reference value.

The candidate compound is a small molecule compound. In some embodiments, the method is used to screen a library of candidate agents, for example, a library of chemical compounds. In some embodiments, the candidate agent comprises a nucleic acid molecule, for example, a DNA molecule, an RNA molecule, or a DNA/RNA hybrid molecule, single-stranded, or double-stranded. In some embodiments, the candidate agent comprises an RNAi agent, for example, an antisense-RNA, an siRNA, an shRNA, a snoRNA, a microRNA (miRNA), or a small temporal RNA (stRNA). In some embodiments, the candidate agent comprises an aptamer. In some embodiments, the candidate agent comprises a protein or peptide. In some embodiments, the candidate agent comprises an antibody or an antigen-binding antibody fragment, e.g., a F(ab′).sub.2 fragment, a Fab fragment, a Fab′ fragment, or an scFv fragment. In some embodiments, the antibody is a single domain antibody. In some embodiments, the agent comprises a ligand- or receptor-binding protein.

Some aspects of this invention provide methods for longitudinal measurement of the growth of the drug-refractory cells or organoids.

Some aspects of this invention provide methods for identifying an effective anti-cancer agent using the drug refractory or diapause like cells or organoids as provided herein. In some embodiments, the method comprises contacting the drug refractory or diapause like cells or organoids as provided herein with candidate agent, for example, by adding the agent to the a culture containing the drug-refractory or diapause like cells or organoids. In some embodiments, the method comprises identifying the candidate agent as an effective anti-cancer agent if the candidate agents inhibits the growth of the drug-refractory or diapause like cells or organoids.

### Definitions

As used herein “2D culture” refers to cells attached directly as monolayer to the plastic or glass surface of a cell culture vessel.

As used herein “stromal sells” refer to fibroblasts with or without other cells and/or elements found in loose connective tissue, including but not limited to, endothelial cells, pericytes, macrophages, monocytes, plasma cells, mast cells, adipocytes, etc.

As used herein “three-dimensional matrix” refers to a three dimensional matrix composed of any material and/or shape that (a) allows cells to attach to it (or can be modified to allow cells to attach to it); and (b) allows cells to grow in more than one layer. This support can also be inoculated with stromal cells to form the three-dimensional stromal matrix.

As used herein, the term “spheroid” or organoid” refers to an aggregate, cluster or assembly of cells cultured to allow three-dimensional growth in contrast to the two-dimensional growth of cells in either a monolayer or cell suspension (cultured under conditions wherein the potential for cells to aggregate is limited). The aggregate may be highly organized with a well-defined morphology or it may be a mass of cells that have clustered or adhered together with little organization reflecting the tissue of origin. It may comprise a single cell type (homotypic) or more than one cell type (heterotypic). Preferably, the cells are primary cells isolated from tissue but may also include cell lines or a combination of primary isolates with an established cell line(s). Particular cell ‘types’ include, but are not limited to, cancer cells and cancer stem cells, somatic cells, stem cells, and progenitor cells.

As used herein, the terms “directly derived” and “patient-derived” refers to cells from a biological tissue and/or cell containing bodily fluid that has been obtained directly from an individual, donor or animal without intermediate steps of subculture in 2D culture conditions. Thus, a suspension of cells is produced directly from the biological tissue and/or cell-containing bodily fluid. This is in contrast to established methods in which stable and highly passaged cell lines in 2D cultures are used. Such cell lines are far removed from being directly derived from their progenitor tissue by several, often a great many, intermediate culture steps. By way of non-limiting example, sources of suitable tissues include, but are not limited to, benign or malignant primary and metastatic tissues, sources of suitable cell containing bodily fluids include, but are not limited to, pleural effusion fluid or ascites fluid (liquid tumors). Preferably the cells originate from a mammal. Ideally the biological tissue sample is a primary isolate tissue sample. Alternatively, the biological tissue sample is an isolate from an intermediate animal host, for example patient-derived cancer xenograft (PDX) in mice.

The term “cell line” as used herein refers to cells derived from a primary culture by subculturing and that have exceeded the Hayflick limit. The Hayflick limit may be defined as the number of cell divisions that occur before a cell line becomes senescent or unable to replicate further. This limit is approximately 50 divisions for most non-immortalized cells and in terms of cell culture, equates to approximately 9 to 10 passages of cell subculture over the course of from about 12 to 14 weeks.

Primary tumors are tumors from the original site where they first developed. For example, a primary brain tumor is one that arose in the brain. This is in contrast to a metastatic tumor that arises elsewhere and metastasized (or spread) to, for example, the brain.

The terms “proliferation” and “expansion” as used interchangeably herein refer to an increase in the number of cells of the same type by division. The term “differentiation” refers to a developmental process whereby cells become specialized for a particular function, for example, where cells acquire one or more morphological characteristics and/or functions different from that of the initial cell type. The term includes both lineage commitment and terminal differentiation processes. Differentiation may be assessed, for example, by monitoring the presence or absence of lineage markers, using immuno-histochemistry or other procedures known to a skilled in the art. Differentiated progeny cells derived from progenitor cells may be, but are not necessarily, related to the same germ layer or tissue as the source tissue of the stem cells. For example, neural progenitor cells and muscle progenitor cells can differentiate into hematopoietic cell lineages.

The term “biomarker” is thereby defined as above and preferably selected from the group of protein biomarkers, molecular biomarkers and genomic biomarkers. “Biomarker” in the context of the present invention encompasses, without limitation, proteins, nucleic acids, and metabolites, together with their polymorphisms, mutations, variants, modifications, subunits, fragments, protein-ligand complexes, and degradation products, protein-ligand complexes, elements, related metabolites, and other analytes or sample-derived measures. Biomarkers can also include mutated proteins or mutated nucleic acids.

Treating” or “treatment” refers to administration of a compound or agent to a subject who has a disorder with the purpose to cure, alleviate, relieve, remedy, delay the onset of, prevent, or ameliorate the disorder, the symptom of the disorder, the disease state secondary to the disorder, or the predisposition toward the disorder. The terms “prevent,” “preventing,” “prevention,” “prophylactic treatment” and the like refer to reducing the probability of developing a disorder or condition in a subject, who does not have, but is at risk of or susceptible to developing a disorder or condition.

A “subject” refers to a human and a non-human animal. In one embodiment, the subject is a human. In another, the subject is an experimental, non-human animal or animal suitable as a disease model. The term “animal” includes all vertebrate animals including humans. It also includes an individual animal in all stages of development, including embryonic and fetal stages. In particular, the term “vertebrate animal” includes, but not limited to, humans, non-human primates (particularly higher primates), canines (e.g., dogs), felines (e.g., cats); equines (e.g., horses), bovines (e.g., cattle), porcine (e.g., pigs), rodent (e.g., mouse or rat), guinea pig, cat, rabbit, as well as in avians, such as birds, amphibians, reptiles, etc. The term “avian” refers to any species or subspecies of the taxonomic class ava, such as, but not limited to, chickens (breeders, broilers and layers), turkeys, ducks, a goose, a quail, pheasants, parrots, finches, hawks, crows and ratites including ostrich, emu and cassowary. Examples of a non-human animal include all non-human vertebrates, e.g., non-human mammals and non-mammals mentioned above.

### OTHER EMBODIMENTS

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

We claim:

1. A method of preparing a drug refractory cell or organoid comprising culturing a population of cells in a three-dimensional cell culture system in the presence of one or more agents capable of inducing death of said cells for a period of time until the longitudinal rate of decreasing cell viability in the culture approximately plateaus, thereby obtaining a drug refractory cell or organoid.

2. A method of preparing cell or organoid in a diapause like state comprising culturing a population of cells in a three-dimensional cell culture system in the presence of one or more agents for a period of time until the longitudinal rate of decreasing cell viability and or growth in the culture approximately plateaus, thereby obtaining a cell or organoid in a diapause like state.

3. The method of claim 2 wherein the cell or organoid in the diapause like state has a gene gene expression signature similar to the state of embryonic diapause

4. The method of claim 3, wherein the gene expression signature is compared by a positive pairwise correlation at pathway, gene ontology (GO)-term or gene level.

5. The method of any one of the preceding claims, wherein the agent is a cytotoxic agent or a targeted therapeutic agent.

6. The method of any one of the preceding claims, wherein the targeted agent is an antibody; a peptide or a nucleic acid.

7. The method according to any one of the proceeding claims wherein the cytotoxic agent is irradiation.

8. The method according to any one of the proceeding claims, wherein the cells are further cultured in the presence of a one or more developmental morphogens.

9. The method of claim 8, wherein said developmental morphogen is a Wnt pathway stimulator.

10. The method of claim 9, wherein the Wnt pathway stimulator is R-spondin or Noggin.

11. The method according to any one of the proceeding claims, wherein the population of cells are transfected with an expression vector encoding a reporter protein prior to culturing in the three-dimensional cell culture system.

12. The method according to any one of the proceeding claims, wherein said cell or organoid is a tumor.

13. The method according to any one of the proceeding claims wherein the populations of cells is a primary tumor cell or a tumor cell line.

14. The method according to any one of the proceeding claims wherein the populations of cells is a tumor of human or animal origin growing in an animal.

15. The cell or organoid produced by any one of the methods of claims 1 to 14.

16. A method of screening a candidate drug, or a candidate drug combination for anti-cancer activity comprising contacting the cell or organoid of claim 15 with said candidate drug or drug combination and determining whether the candidate induces cell or organoid death or inhibits cell or organoid growth.

17. The method of claim 16, wherein death or inhibition of growth of the cell or organoid is determined by a cell viability assay, a cell reporter assay, or a microscopic assay.

18. The method of claim 16 or 17, wherein the candidate drug combination is administered concomitantly or sequentially.

19. A method of longitudinally simulating the relapse of residual cancer in a subject comprising longitudinal measurement of growth of the cell or organoid of claim 15.

20. The method of claim 19, wherein growth of the cell or organoid is determined by a cell viability assay, a cell reporter assay, or a microscopic assay.

21. A method of screening a candidate drug, or a candidate drug combination capable of treating residual cancer in a subject comprising contacting the cell or organoid of claim 12 with said candidate drug or drug combination and determining whether the candidate induces cell or organoid death or inhibits cell or organoid growth.

22. The method of claim 21, wherein the cell or organoid is autologous to the subject.

23. The method of claim 21, wherein death or inhibition of growth of the cell or organoid is determined by a cell viability assay, a cell reporter assay, a microscopic assay.

24. The method of claims 21-23, wherein the candidate drug combination is administered concomitantly or sequentially.

25. A cancer vaccine comprising the cell or organoid of claim 15 or portion thereof, wherein said cell or organoid is dead, irradiated and/or modified to express an immune-stimulatory factor.

26. A method of identifying a biomarker associated with a drug refractory tumor, comprising comparing a gene or protein expression profile obtained from the cell or organoid of claim 15, with a reference gene or protein expression profile obtained from cell population used to produce the cell or organoid

27. A regenerative medicine method comprising the use of the cell or organoid of claim 15 when the said cell or organoid contains desirable gene expression, epigenetic, and/or stem cell-like properties.

28. A method of screening a candidate drug, or a candidate drug combination capable of reversing the drug refractory state or the diapause-like state comprising:
a. contacting the cell or organoid of claim 12 with said candidate drug or drug combination and one or more agents capable of inducing death of said cell; and
b. determining if cell or organoid death occurs,
c. thereby identifying a candidate drug, or a candidate drug combination capable of reversing the drug refractory state or diapause like state
